(19) United States
(12) Patent Application Publication
(10) Pub. No.: US 2006/0211976 A1
(43) Pub. Date: Sep. 21, 2006

(54) Self-applied pressure dressing

(76) Inventor: Troy William Ramsey, Colorado Springs, CO (US)

Correspondence Address: CRAIG OLOFSON, P O BOX 2386, LOS GATOS, CA, 95031, US

(21) Appl. No.: 11/026,453

(22) Filed: Dec. 29, 2004

(51) Int. Cl. A61L 15/00

(52) U.S. Cl. 602/75

(57) ABSTRACT

A loop shaped bandage consisting of a strap (11), a squared oval (12) a handle loop (13), and multiple strips hook (14) and loop (15) fasteners.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

1. Field of Invention

This invention relates to bandages, specifically to a bandage to control the bleeding of a wound to an extremity in an emergency pre-hospital or military field environment.

2. Prior Art

Previously bandages were constructed of simple strips of cloth with the ends of the strips being wrapped around the wounded area of the body. The bandage was held in place by the ends being tied into a knot. This required the use of both hands, and was impossible to accomplish if one had to treat a wound on their own arm.

Thereafter several improvements have been made to bandaging material. The improvements include; the use of an absorbent pad to place on the wound site, the sterilization of the bandaging material, the use of various mechanical fasteners, the use of elastic materials, the use hook and loop fastener as a closure, the use of inflatable bladders. But all of the improvements have failed to produce a bandage that is simply and quickly applied with one hand.

### OBJECTS AND ADVANTAGES

Accordingly, several objects and advantages of the invention are:
(a) to provide a bandage that can be easily and rapidly applied with one hand;
(b) to provide a bandage simple enough that it can be used by non-medical personnel with a minimum of training;
(c) to provide a bandage capable of providing variable pressure, with a range from minor pressure for treating simple lacerations, to tourniquet like pressure for treating wounds of the severity of traumatic amputation;
(d) to provide a single bandage capable of treating wounds on limbs of wide variety of sizes, from small children, to well developed adults;
(e) to provide a bandage with a simple and secure closure.

Further objects and advantages will become apparent from a consideration of the ensuing description and drawings.

### SUMMARY

In accordance with the invention, the body of the bandage is a strap of cloth with strips of hook and loop fasteners attached perpendicular to the long axis of the bandage. At one end of the strap a squared oval is attached to the bandage strap. The squared oval creates a cinching point. The second end of the strap it passed through the squared oval making the bandage into a loop. The second end of the strap has a smaller loop attached to it, to create an easy to grab handle to cinch the bandage tight. The handle loop is attached in such a way as to make it difficult for the handle loop to pass through the squared oval, ensuring that the bandage is likely to remain in a loop.

### DRAWINGS—FIGURES

FIG. 1 shows the bandage, fully assembled, from a side view, as it would look before it was applied.

FIG. 2 shows the bandage, fully assembled, from a side view, as it would look after it was applied.

FIG. 3 shows the bandage, laid flat, from a top view.

FIG. 4 shows the bandage, laid flat, from a side view.

### DRAWINGS—REFERENCE NUMERALS

11 bandage strap
12 squared oval
13 handle loop
14 hook portion of hook and loop fastener
15 loop portion of hook and loop fastener
16 wounded limb
17 attachment strap
18 sewn stitching

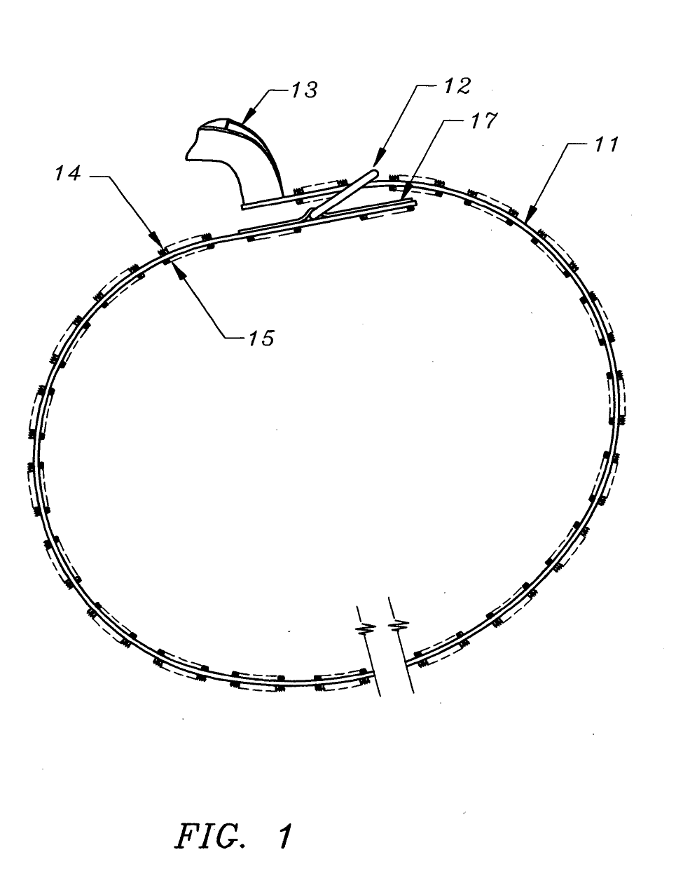

### DETAILED DESCRIPTION—FIGS. 1,2,3 AND 4—PREFERRED EMBODIMENT

The following represents my preferred embodiment at this time.

Bandage strap 11 is a 2 to 3 inch wide strap of elastic cloth, 36 to 48 inches long.

Squared oval 12 is made of plastic or metal. Squared oval 12 is one half inch longer than bandage strap 11 is wide, and wide enough for bandage strap 11, when assembled with hook 14, and loop 15 portions of the hook and loop fastener, to slide readily through squared oval 12, but narrow enough that it is difficult to pass handle loop 13 through squared oval 12. Squared oval 12 is secured to bandage strap 11 by sewing attachment strap 17 to bandage strap 11.

Handle loop 13 is made of an approximately eight inch long piece of 1 inch wide tubular nylon. One end of nylon is sewn to bandage strap 11. A loop with a twist is formed, by putting a half turn in handle loop 13, then sewing remaining end of handle loop 13 adjacent to the first sewn portion on bandage strap 11.

Each hook portion 14 of the hook and loop fastener is sized as long as bandage strap 11 is wide, and three quarters of an inch wide. Multiple hook portions 14 are sewn to bandage strap 11 on the outside of bandage strap 11 loop and perpendicular to the long axis of bandage strap 11. These hook portions 14 are spaced approximately one half inch apart and sewn the entire length of bandage strap 11, less the portion of bandage strap 11 where attachment strap 17 is sewn.

Each loop portion 15 of the hook and loop fastener is sized as long as bandage strap 11 is wide, and three quarters of an inch wide. Multiple loop portions 15 are sewn to bandage strap 11 on the inside of bandage strap 11 loop and perpendicular to the long axis of bandage strap 11. These loop portions 15 are spaced approximately one half inch apart and sewn the entire length of bandage strap 11.

This spacing of multiple portions hook 14 and loop 15 fasteners allows the elastic cloth of bandage strap 11 to retain its elasticity while ensuring a secure closure. Both the hook 14 and loop 15 portions have their edges sewn down, to ease sliding through squared oval 12.

Attachment strap 17 is made of non-elastic cloth as wide as bandage strap 11, and approximately 2 inches long. Attachment strap 17 is run through squared oval 12 and both ends are sewn to bandage strap 11, with squared oval 12 in between the sewn ends.

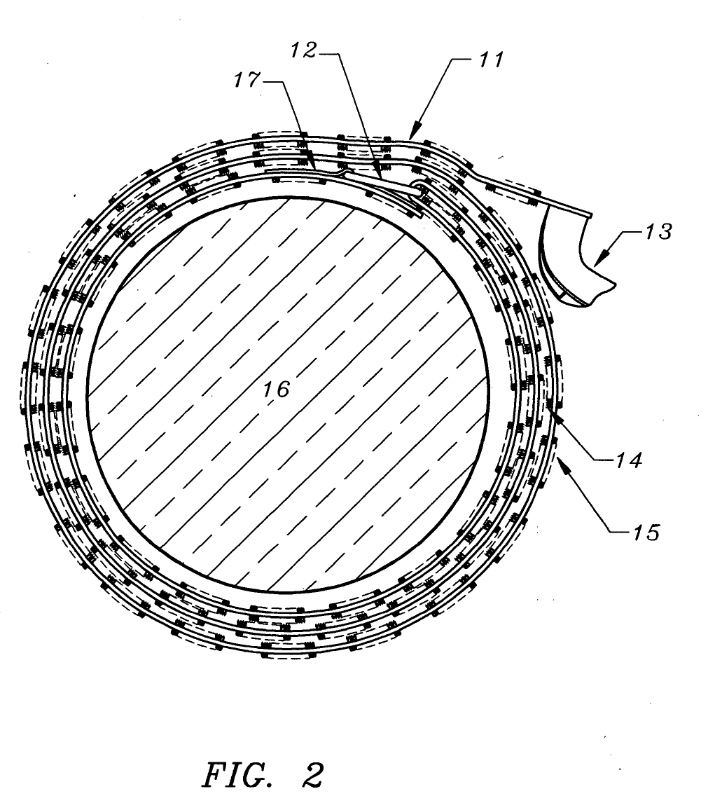

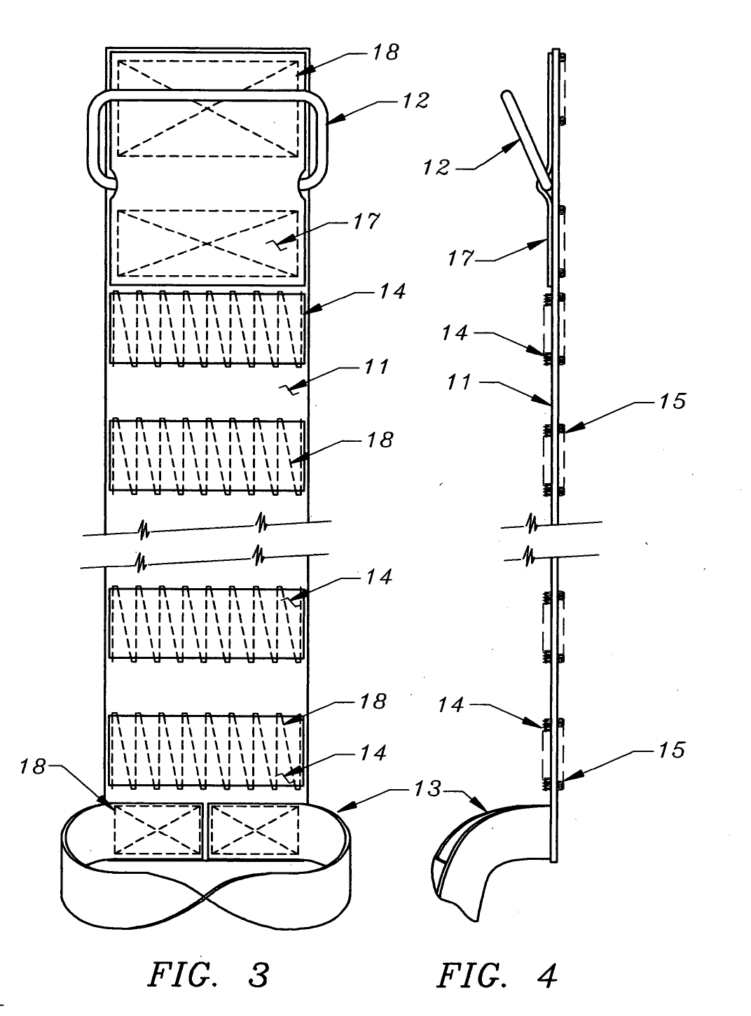

### OPERATION—FIGS. 1 AND 2

The bandage is used by placing the injured limb into the loop of bandage strap 11. The bandage is placed directly on the wound for use as a pressure dressing, or proximal (i.e. closer to the body than the wound) on the injured limb for use as a tourniquet. Handle loop 13 is pulled away from the limb to tighten the loop of bandage strap 11. Squared oval 12 provides a mechanical advantage in applying pressure until the bleeding is controlled. The loose end of bandage strap 11 is wrapped back around the loop continuing to apply pressure and the hook 14 and loop 15 fasteners are used to secure the dressing in place.

### ADVANTAGES

From the description above, a number of advantages of my self-applied pressure dressing become evident:
(a) Since the bandage remains in the shape of a loop, and there aren't two ends of the bandage, it can easily be applied with one hand.
(b) The use of a single, simple pull loop can greatly decrease the time required to use this bandage.
(c) Once pressure is applied, it is easily maintained and may even be increased, using the elasticity of the bandage.
(d) The bandage is simple to use, requiring minimal training for use by non-medical personnel.
(e) The use of multiple strips of hook and loop fasteners, allows the bandage to be used on limbs of a wide variety of sizes.
(f) The sizing of the hook and loop fasteners, and use of multiple fasteners, ensure a secure closure of the bandage.
(g) Being simple to manufacture and still very effective, this bandage is very economical.
(h) A bandage that can be simply and rapidly applied in an emergency situation, will save lives, and this is such a bandage.

### CONCLUSION, RAMIFICATIONS, AND SCOPE

Thus the reader will see that the bandage of the invention provides a simple, inexpensive, easily applied, life saving device.

While my above description contains many specificities, these should not be construed as limitations on the scope of the invention, but rather as an exemplification of one preferred embodiment thereof. For example an absorbent pad may be attached to the inside of the bandage. Another example is an additional squared oval or similar device may be attached elsewhere on bandage strap, in order to provide additional mechanical advantage in tightening the bandage. Another example is that the spacing and dimensions of the hook and loop fasteners may be changed. Another example is that attachment strap can be omitted, in favor of securing the oval by turning the main strap of the bandage back on itself, through the oval. A final example would be the substitution of non-elastic cloth for elastic cloth in the main body of the bandage.

Accordingly, the scope of the invention should be determined not by the embodiment illustrated, but by the appended claims and their legal equivalents.

I claim:

1. A bandage for treating wounds to an extremity comprising:
a. A strap of material,
b. A means for cinching on the first end of said strap,
c. The second end of said strap being routed through said means for cinching, forming a loop of said strap,
d. A handle on the second end of said strap, where pulling said handle makes said loop smaller, applying pressure on an extremity in said loop,
e. A means of securing the second end of said strap, where said pressure inside said loop is maintained.
